(19) United States
(12) Patent Application Publication
(10) Pub. No.: US 2005/0010225 A1
(43) Pub. Date: Jan. 13, 2005

(54) Plate for osteosynthesis having variable flexibility

(76) Inventor: Nilli Del Medico, Orbassano (IT)

Correspondence Address: John M Siragusa, 400 W Maple, Suite 350, Birmingham, MI, 48009, US

(21) Appl. No.: 10/494,894

(22) PCT Filed: Sep. 11, 2002
(86) PCT No.: PCT/IB02/03742

(30) Foreign Application Priority Data
Nov. 9, 2001 (IT) T02001A001059

(51) Int. Cl. A61F 2/30; A61B 17/56

(52) U.S. Cl. 606/69

(57) ABSTRACT

A plate for osteosynthesis, including a body of elongate form provided with a plurality of holes for the passage of corresponding screws for the fixing to a fractured bone. The body of elongate form includes a portion of reduced section defining a an intermediate point having an increased flexibility. The portion of reduced section coupled with a mobile portion of complementary shape acting from the exterior of the plate, to constrain the mobile portion to the body of the plate in order to decrease the flexibility of the intermediate point.

The present invention relates to a plate for osteosynthesis.

More particularly, the invention relates to a plate having an elongate shape provided for being applied, by means of suitable screws, to a fractured bone of elongate type.

The plates of this type are provided over their length with a plurality of holes for the passage of corresponding screws, and are available in different models, depending on the shape and size of the bone they are to be applied to.

The plates of elongate type currently used are characterised by a constant and quite high structural rigidity, which is certainly necessary for assuring a correct approach of the bone sections during the initial period, but which can turn out to be excessive in the successive periods, that is when the fracture results to be already partly mended and the bone can be already subjected to external stresses.

It is an object of the invention to provide a plate for osteosynthesis which is equipped with a system allowing to vary the flexibility of the plate, also after it has been applied.

The above and other objects are achieved by means of the plate for osteosynthesis made in accordance with the invention, as claimed in the hereby attached claims.

The plate, made in accordance with the invention, is preferably of elongate type and can be manufactured in different shapes and sizes to suit to different types of bones and fractures.

The above and other objects of the invention will become more readily apparent from the following description of a preferred embodiment, with reference to the accompanying drawings, in which:

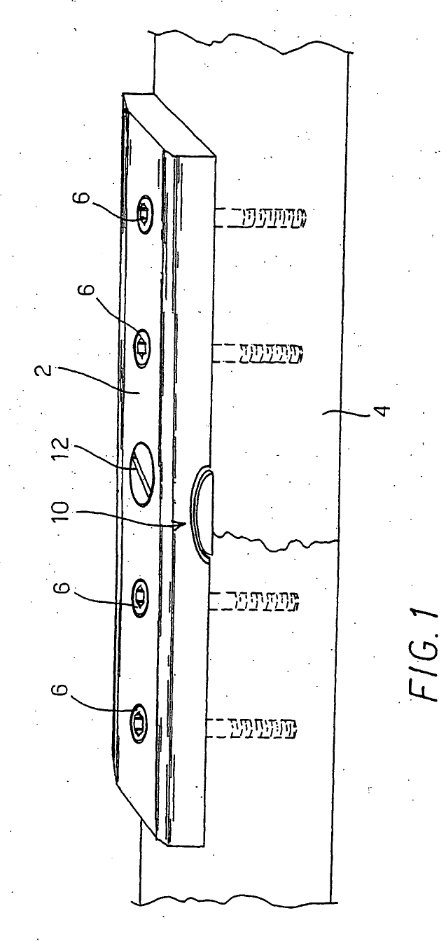

FIG. 1 is a perspective view of a plate for osteosynthesis applied to a fractured bone, made in accordance with the invention;

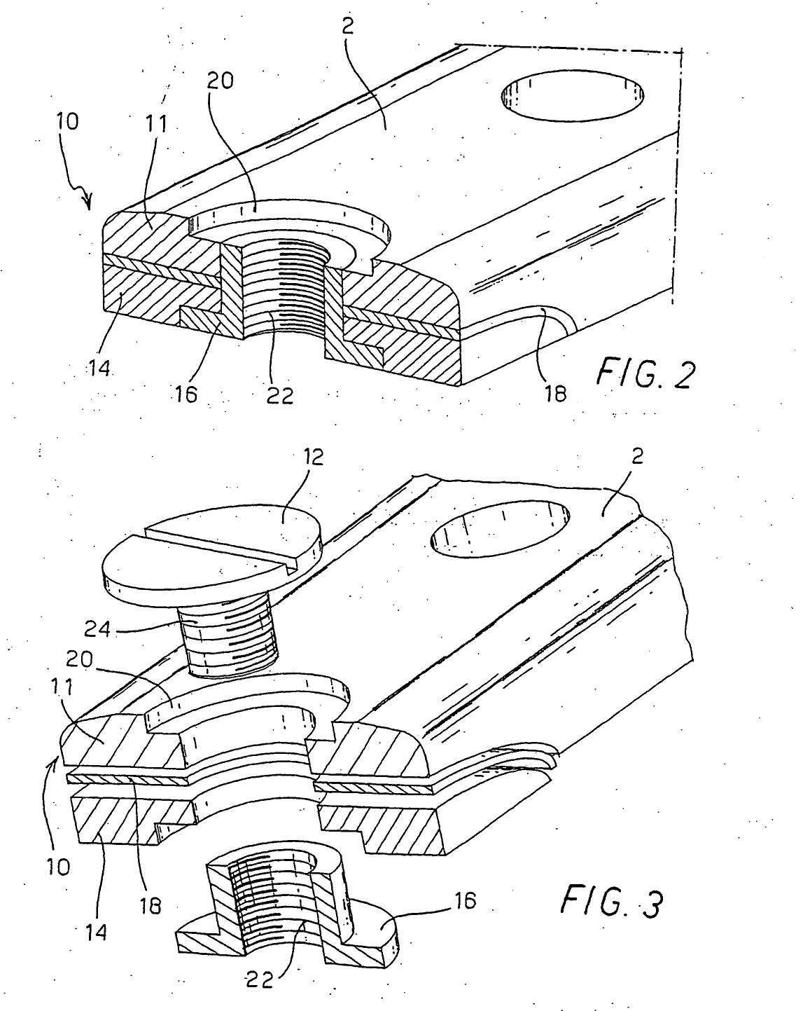

FIG. 2 is a partial section of a perspective view of a particular of the plate for osteosynthesis of FIG. 1;

FIG. 3 is an exploded perspective view of the particular of FIG. 2; and

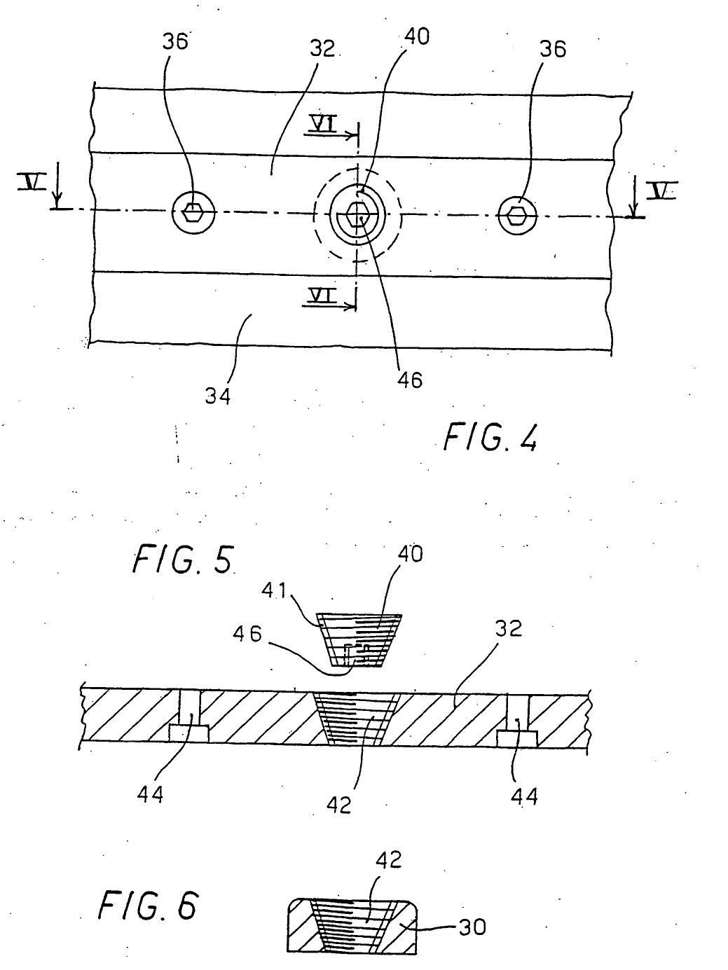

FIG. 4 is a top view of a second embodiment of a plate for osteosynthesis made in accordance with the invention;

FIG. 5 is a sectional side view of the plate for osteosynthesis of FIG. 4; and

FIG. 6 is a cross-sectional view of the plate for osteosynthesis of FIG. 4.

Referring to FIG. 1, a plate for osteosynthesis comprises a body 2 having an elongate shape and is provided with a plurality of holes for the passage of corresponding screws 6 to be fixed to a fractured bone 4. Said plate is provided, at an intermediate point 10, with a portion having a reduced section coupled with a mobile portion of complementary shape.

The portion of reduced section defines an intermediate point having an higher flexibility, while the mobile portion allows to vary or even to nullify said flexibility thanks to a system that will be successively described with reference to the FIGS. 2 and 3.

The FIGS. 2 and 3 show in detail the point 10, having variable flexibility, of the elongate body 2 of the plate. The portion 11 of reduced section is obtained in the plate for instance by removing a portion of material of the desired thickness through a transversal cut.

The same portion that has been removed can be used as a mobile portion 14, preferably by interposing an intermediate layer 18 made in a plastic material, for instance Teflon, in order to avoid the direct contact between metal and metal.

The mobile portion 14 is coupled with the plate by means of a screw-adjustable system that can be actuated from the side of the plate opposite to the side facing the bone. In particular, the mobile portion 14 comprises an internally threaded 22 pin 16, inserted into a through hole, that is coupled with the threaded shank 24 of a screw 12. The screw 12 is provided with a head that, in order not to project from the surface of the plate, is inserted into a suitable seat 20.

By screwing the screw 12 into the pin 16, the mobile portion 14 is pressed against the corresponding seat obtained in the plate, thereby providing the plate itself with the maximum of rigidity. On the contrary, by progressively unscrewing the screw 12, it is possible to decrease gradually the rigidity of the plate in the point 10, thereby increasing its flexibility.

The screw 12 allows to adjust the flexibility of the plate both before it is applied to the bone, when high rigidity is generally required, and successively, simply by making a small cut on the skin and acting on the screw by means of a suitable tool.

The Figures from 4 to 6 show a second embodiment of a plate for osteosynthesis made according to the present invention.

A plate 32, equipped with holes 44 for inserting screws 36 for the fixing to a bone 34, is provided at an intermediate point with a portion 30 of reduced section which increases the flexibility of said plate.

The portion 30 of reduced section is obtained by making in the plate a frustoconical threaded hole 42 having its major base on the side of the plate facing the bone 34.

Inside the threaded hole 42 is screwed a frustoconical screw 40 having its body 41 externally threaded and an hexagonal recess 46 in correspondence with its minor base. By inserting a screwdriver tool in the recess 46, it is possible, from the side of the plate opposite to the bone, to screw or unscrew the screw 40 in the seat 42, thereby varying the flexibility of the plate itself in that point.

When the screw 40 is tightened in its seat 42, the plate presents the maximum of rigidity: on the contrary, when the screw is unscrewed, also slightly, the maximum of flexibility is obtained. Preferably, the screw 40 is of a length slightly smaller than the thickness of the plate, in order not to interfere with the bone 34 when said screw is unloosed.

1. A plate for osteosynthesis, comprising a body of elongate shape, provided with a plurality of holes for the passage of corresponding screws for the fixing to a bone wherein said body of elongate shape, includes a portion of reduced section having a cross-sectional area smaller than the cross-sectional area of said body of elongate shape to define an intermediate point, said intermediate point having a greater flexibility than said body of elongate shape, said portion of reduced section being coupled with a mobile portion of complementary shape provided with an adjustable member adjustable from the exterior of said plate, to constrain said mobile portion to said body of elongate shape in order to decrease the flexibility of said intermediate point.

2. The plate for osteosynthesis according to claim 1, wherein said mobile portion is a portion obtained by removing a part of the thickness from the plate itself through a transversal cut.

3. The plate for osteosynthesis according to claim 1, wherein said adjustable member comprises a screw, said screw provided with a threaded body that engages into an internally threaded pin inserted into a through hole in said mobile portion.

4. The plate for osteosynthesis according to claim 3, wherein said screw is provided with a head that is inserted into a corresponding seat obtained in said plate, and said pin is collapsibly inserted into said mobile portion.

5. The plate for osteosynthesis according to claim 1, wherein a layer of plastic material is present between said mobile portion and the corresponding portion of reduced section of the body of elongate shape.

6. The plate for osteosynthesis according to claim 5, wherein said layer of plastic material is a layer of Teflon.

7. The plate for osteosynthesis according to claim 1, wherein said portion of reduced section is obtained in said body of elongate shape by making a frustoconical internally threaded through hole and said mobile portion is an externally threaded frustoconical screw of shape complementary to said frustoconical hole.

8. The plate for osteosynthesis according to claim 7, wherein said frustoconical screw is provided, in correspondence with its minor base, with a seat for inserting a suitable screwdriver tool.

9. The plate for osteosynthesis according to claim 8, wherein said seat is accessible from a side of the plate opposite to the side facing the bone to which the plate is applied.
